(19) United States
(12) Patent Application Publication
(10) Pub. No.: US 2008/0262266 A1
(43) Pub. Date: Oct. 23, 2008

(54) Method for the Production of Xylyendiamine

(75) Inventors: Kirsten Dahmen, Mannheim (DE); Sabine Huber, Limburgerhof (DE); Randolf Hugo, Dirmstein (DE); Johann-Peter Melder, Bohl-Iggelheim (DE); Thomas Preiss, Weisenheim (DE)

Correspondence Address: CONNOLLY BOVE LODGE & HUTZ LLP, 1875 EYE STREET, N.W., SUITE 1100, WASHINGTON, DC, 20036, US

(73) Assignee: BASF SE, Ludwigshafen (DE)

(21) Appl. No.: 12/067,893

(22) PCT Filed: Sep. 14, 2006
(86) PCT No.: PCT/EP2006/066342

§ 371 Date: Mar. 24, 2008

(30) Foreign Application Priority Data
Sep. 24, 2005 (DE) 10 2005 045 806.8

(51) Int. Cl. C07C 209/48; C07C 211/27

(52) U.S. Cl. 564/305; 564/415

(57) ABSTRACT

Process for preparing o-, m- or p-xylylenediamine by hydrogenation of o-, m- or p-phthalonitrile in the presence of a heterogenous catalyst, which comprises feeding a solution of the phthalonitrile in the corresponding isomer of crude xylylenediamine into the hydrogenation reactor, with the crude xylylenediamine having a purity in the range from 85 to 99.7% by weight and a content of higher boilers in the range from 0.3 to 15% by weight.

The present invention relates to a process for preparing xylylenediamine by hydrogenation of phthalonitrile in the presence of a heterogeneous catalyst.

Xylylenediamine(bis(aminomethyl)benzene) is a useful starting material, e.g. for the synthesis of polyamides, epoxy hardeners or as intermediate for preparing isocyanates.

The synthesis of xylylenediamine by hydrogenation of phthalonitrile is known.

The term “xylylenediamine” (XDA) comprises the three isomers ortho-xylylenediamine, meta-xylylenediamine (MXDA) and para-xylylenediamine.

The term “phthalonitrile” (PN) comprises the three isomers 1,2-dicyanobenzene=o-phthalonitrile, 1,3-dicyanobenzene=isophthalonitrile=IPN and 1,4-dicyanobenzene=terephthalonitrile.

The phthalonitriles are solids (e.g. isophthalonitrile (IPN) melts at 161° C.) and have relatively poor solubilities in organic solvents.

The literature teaches mainly alcohols, amides, cyclic ethers or amines as solvents for the hydrogenation of nitriles to primary amines.

EP-A1-1 209 146 (BASF AG) relates to a process for the hydrogenation of nitrites to primary amines over specific Raney catalysts. Solvents mentioned are alcohols, amines, amides such as NMP and dimethylformamide (DMF), ethers and esters.

WO-A-98/09947 (Du Pont) describes the hydrogenation of 2-methylglutaronitrile in the presence of numerous possible solvents, including NMP (cf. claim 2).

JP-A-2002 205980, WO-A-2000/046179, JP-A-54 041 804 and JP-B-54 037 593, for example, describe the use of alcohols, in particular-methanol, as solvents for the hydrogenation of PN.

A disadvantage of the use of methanol (solubility of IPN at 60° C. 18% by weight) is that methylated XDA occurs as by-product.

CN-A-1 285 343 (Derwent Abstract WP2001317563) (China Petrochem. Corp.) describes the use of amines as solvents for the hydrogenation of PN.

U.S. Pat. No. 4,482,741 (UOP) describes the use of MXDA as solvent. The solubility of IPN in MXDA at 70° C. is about 20% by weight. However, large purification streams of MXDA are necessary here. For example, a 20% strength solution of IPDN in pure MXDA requires 5 times the purification capacity which would be required for the purification of the product formed alone. Capital and operating costs are correspondingly higher.

EP-A-538 865 and U.S. Pat. No. 4,247,478-teach the use of ethers such as dioxane, THF and diglyme as solvents for the hydrogenation of PN.

Although the solubility of IPN in THF of barely 19% by weight at 60° C. is satisfactory, a disadvantage of ethers as solvents is their tendency to form undesirable peroxides.

EP-A2-1 193 247 and EP-A1-1 279 661 (both Mitsubishi Gas Chem. Comp.) relate to a process for purifying isophthalonitrile (IPN) or a process for preparing pure XDA.

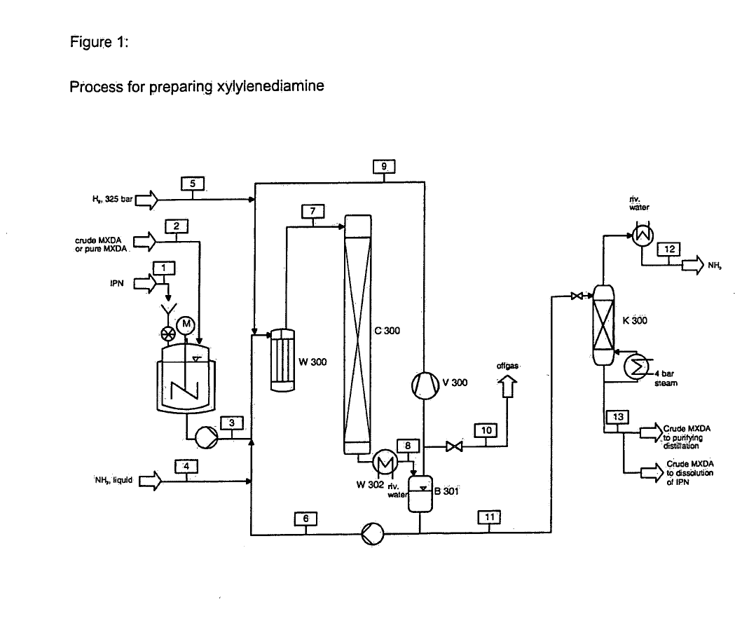

EP-A2-1 193 247 discloses the hydrogenation of IPN in the presence of NH₃ and a solvent (cf. FIG. 1).

EP-A1-1 279 661 discloses aromatic hydrocarbons and saturated hydrocarbons as solvents for the hydrogenation (column 7, paragraph [0038]).

EP-A2-1 193 244 (Mitsubishi Gas Chem. Comp.) describes a process for preparing XDA by hydrogenation of phthalonitrile dissolved in a C₆-C₁₂ aromatic hydrocarbon such as xylene, mesitylene and pseudocumene (columns 5-6, paragraphs, [0027] and [0028]; column 6, paragraph [0032]).

U.S. Pat. No. 3,069,469 (California Research Corp.) teaches the use of aromatic hydrocarbons, xylene, dioxane and aliphatic alcohols as solvents for the hydrogenation of aromatic nitrites such as PN.

DE-A-21 64 169 (Mitsubishi Gas Chem. Comp.) describes, on page 6, last paragraph, the hydrogenation of IPN to MXDA in the presence of an Ni and/or Co catalyst in ammonia as solvent.

GB-A-852,972 (equivalent: DE-A-11 19 285) (BASF AG), too, discloses the use of ammonia as solvent in the hydrogenation of PN.

The eight patent applications WO-A-05/028417, WO-A-05/026102, WO-A-05/026103, WO-A-05/026104, WO-A-05/026100, WO-A-05/026101, WO-A-05/026098 and WO-A-05/026099 (each BASF AG) each relate to processes for preparing XDA.

The German patent application no. 102005036222.2 of Aug. 2, 2005 (BASF AG) relates toga process for preparing xylylenediamine by continuous hydrogenation of phthalonitrile over a heterogeneous catalyst in the presence of liquid ammonia in a reactor, with part of the output from the reactor being continuously recirculated as liquid recycle stream to the reactor inlet (recycle mode), in which phthalonitrile is taken off as a melt or in solid form with a stream of liquid ammonia (stream a) and a further stream which is taken off at least as substream from the recycle stream around the hydrogenation reactor (stream b) by means of a mixing device or a mixture of the streams a and b is mixed and the resulting liquid mixture is fed into the hydrogenation reactor.

The handling of liquid ammonia solvent and solutions in ammonia requires special pressure apparatuses which are not always available.

It is an object of the present invention to discover an improved economical process for preparing high-purity xylylenediamine, in particular meta-xylylenediamine, in high yield and space-time yield (STY), which can be carried out at throughputs comparable to those in processes of the prior art. The introduction of the nitrile or its solution into the hydrogenation reactor should be able to take place at moderate temperatures (e.g. ≦80° C.) and pressures (e.g. ≦0.6 bar) and the outlay for distillation should be kept as small as possible so that the preparation of XDA can be carried out in existing plants or standard apparatuses without the need for capital investment.

We have accordingly found a process for preparing o-, m- or p-xylylenediamine by hydrogenation of o, m- or p-phthalonitrile in the presence of a heterogeneous catalyst, which comprises feeding a solution of the phthalonitrile in the corresponding isomer of crude xylylenediamine into the hydrogenation reactor, with the crude xylylenediamine having a purity in the range from 85 to 99.7% by weight and a content of higher boilers in the range from 0.3 to 15% by weight.

Preference is given to feeding a solution of the phthalonitrile in the corresponding isomer of crude xylylenediamine which has a purity in the range from 89 to 99.5% by weight, in particular in the range from 92 to 99.2% by weight, and a content of higher boilers in the range from 0.5 to 11% by weight, in particular in the range from 0.8 to 8% by weight.

The higher boilers are, for example, amides, amidines, bisXDA (XDA dimers) and further oligomers, e.g. compounds of the following formulae:

amides: e.g.

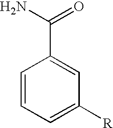

R═—CH₂NH₂, —CN, —CONH₂, —CH₂NHCH₂-aryl, —C(NH)NCH₂-aryl, —CHNCH₂-aryl

amidines: e.g.

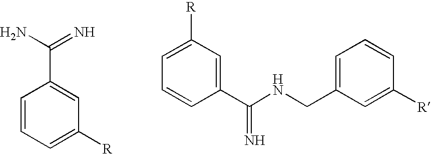

R, R′(independently of one another)=—CH₂NH₂, —CN, —CONH₂, —CH₂NHCH₂-aryl, —C(NH)NCH₂-aryl, —CHNCH₂-aryl

bisXDA: e.g. bisMXDA

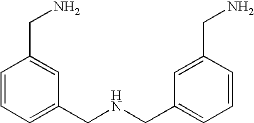

Other oligomers: e.g.

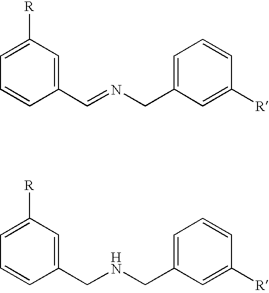

R, R′(independently of one another)=—CH₂NH₂, —CN, —CONH₂, —CH₂NHCH₂-aryl, —C(NH)NCH₂-aryl, —CHNCH₂-aryl

The crude xylylenediamine used as solvent preferably has a content of lower boilers such as benzylamine and/or N-methylbenzylamine in the range from 0.01 to 2% by weight, particularly preferably in the range from 0.01 to 1% by weight, (in each case without ammonia) and an ammonia content in the range from 0 to 5% by weight, particularly preferably in the range from 0 to 2% by weight.

“Higher boilers” are components which under the same conditions have a boiling point higher than that of the respective xylylenediamine.

“Lower boilers” are compounds which under the same conditions have a boiling point lower than that of the respective xylylenediamine.

The isomer of XDA corresponding to m-phthalonitrile (=isophthalonitrile) is meta-XDA. An analogous situation applies to, the other isomers.

In the work-up, it is important not to use any additional solvent since this would increase the costs for distillation and logistics. Logistically, the reuse of the solvent would be quite complicated, in particular when relatively small amounts of XDA are to be prepared. In any case, however, further materials costs would be incurred for the solvent. In addition, care has to be taken to keep the number of plants and subunits occupied and also their size (and logistics) as small as possible. This is achieved according to the invention when XDA obtained from the hydrogenation is used as crude product, i.e. without further work-up, as solvent.

The process of the invention is preferably employed for preparing meta-xylylenediamine (MXDA) by hydrogenation of isophthalonitrile (IPN).

It is known that MXDA is suitable as solvent for IPN. Owing to the poor solubility (e.g. 15% by weight at 60° C.), this makes high distillation capacities necessary. According to the invention, it has been recognized that the use of the crude MXDA obtained (reaction product mixture after removal of any ammonia used in the reaction) enables the distillation streams to be greatly reduced (virtually the same amount as IPN used as feed stream for the purifying distillation). The reaction product mixture comprises by-products of the reaction (e.g. benzylamine, methylbenzylamine, methylated MXDA, amides, amidines, bisMXDA, further high boilers) and possibly residual amounts of ammonia.

However, the recirculation of the crude MXDA for the dissolution of IPN leads to accumulation of secondary components, in particular those having a boiling point higher than that of MXDA. Astonishingly, no decrease in the catalyst activity or selectivity was observed even up to an accumulation of more than 10% by weight of high boilers in the crude MXDA at a space velocity over the catalyst of 0.3 kg/I/h.

The PN used as starting material in the process can be synthesized in a preceding stage by ammonoxidation of the corresponding xylene isomer. Such syntheses are described, for example, in the BASF patent applications EP-A-767 165, EP-A-699 476, EP-A-222 249, DE-A-35 40 517 and DE-A-37 00 710, in the applications EP-A2-1 193 247, EP-A1-1 279 661 and EP-A2-1 193 244 (all Mitsubishi Gas-Chem. Comp.) mentioned at the outset and in the abovementioned BASF patent applications for the preparation of XDA.

The process of the invention can be carried out as follows:

For the hydrogenation of the phthalonitrile to the corresponding xylylenediamine (o-, m- or p-xylylenediamine) according to the equation

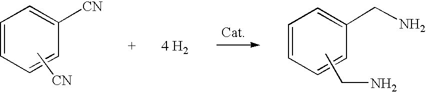

the PN is dissolved in crude XDA. This can be carried out, for example, separately, i.e. in a preceding step, in a container or stirred vessel which is operated batchwise, semi-continuously or continuously and may, if appropriate, have an external pumped circuit, or another suitable mixing or dissolution apparatus.

To increase the rate of dissolution and/or to increase the amount of dissolved PN, the dissolution step can be carried out at elevated temperature, e.g. at from 40 to 120° C., preferably from 50 to 80° C., particularly preferably from 55 to 70° C. The heat can be introduced via a double wall, heating coils, external heat exchangers or another facility suitable for heat transfer. The dissolution step is preferably carried out at an absolute pressure in the range from 1 to 20 bar, preferably from 1 to 6 bar.

Preference is given to using from 7.5 to 25% strength by weight, in particular from 10 to 20% strength by weight, solutions of PN, in particular IPN, in the crude XDA in the process of the invention.

The accumulation of relatively large amounts of by-products can be controlled by regular continuous discharge of crude XDA, e.g. crude MXDA. It is advantageous to correlate the amount of material discharged with the amount of PN, e.g. IPN, introduced. In this way, the use of pure XDA, e.g. MXDA, is necessary only at the beginning of a campaign. This allows the distillation streams, apart from these first input amounts, to be reduced to just the XDA formed. In the other case, i.e. when pure XDA is used in place of the crude XDA for dissolving the PN, the use of a, for example, −15% strength by weight solution would result in 7 times the amount of XDA to be distilled.

However, depending on technical possibilities, continuous discharge of relatively large amounts of XDA can be advantageous.

In the case of a large-accumulation of by-products, it can be necessary to use at least small amounts of distilled XDA as solvent after a certain number of cycles.

However, in all cases the outlay for distillation is many times smaller than when using a solvent or in the case of the exclusive use of purified XDA.

For the hydrogenation of the phthalonitrile to the corresponding xylylenediamine (o, m- or p-xylylenediamine), ammonia, preferably in liquid form, is particularly preferably added to the solution.

The weight ratio of dinitrile to ammonia in the fresh feed is generally from 1:0.15 to 1:15 preferably from 1:0.5 to 1:10, in particular from 1:1 to 1:5.

For the hydrogenation, it is possible to use the catalysts and reactors (e.g. fixed-bed or suspension mode) and processes (continuous, semicontinuous (semibatch), discontinuous (batch)) known to those skilled in the art for this reaction.

In the fixed-bed catalyst mode, both the upflow mode and the downflow mode are possible. Preference is given to the downflow mode.

The hydrogenation reactor can be operated in a single pass. Asian alternative, a recycle mode in which part of the output from the reactor is recirculated to the reactor inlet is also possible. According to one of the preferred embodiments, the process is carried out continuously and part of the stream from the reactor is recirculated continuously as a liquid recycle stream to the reactor inlet (recycle mode). This makes it possible to achieve optimal dilution of the reaction solution, which has a favorable effect on the selectivity. In particular, the recycle stream can be cooled in a simple and inexpensive manner by means of an external heat exchanger and the heat of reaction can be removed in this way. The reactor can as a result be operated adiabatically, with the temperature increase in the reaction solution being able to be limited by means of the cooled recycle stream. Since the reactor does not have to be cooled in this case, a simple and inexpensive construction is, possible. An alternative is a cooled shell-and-tube reactor.

As catalysts, it is possible to use the heterogeneous catalysts known in the prior art for the hydrogenation of aromatic nitriles.

Preference is given to catalysts comprising cobalt and/or nickel and/or iron, as all-active catalyst or on an (inert) support.

Suitable catalysts are, for example, Raney nickel, Raney cobalt, all-active Co catalyst, titanium-doped cobalt on a support (JP-A-2002 205980), Ni on an SiO₂ support (WO-A-2000/046179), Co/Ti/Pd on an SiO₂ support (CN-A-1 285 343, CN-A-1 285 236) and nickel and/or cobalt on a zirconium dioxide support (EP-A1-1 262 232).

Particularly preferred catalysts are the all-active cobalt catalysts doped with Mn, P and alkali metal (Li, Na, K, Rb, Cs) which are disclosed in EP A1-742 045 (BASF AG). The catalytically active composition of these catalysts comprises, prior to reduction with hydrogen, from 55 to 98% by weight in particular from 75 to 95% by weight, of cobalt, from 0.2 to 15% by weight of phosphorus, from 0.2 to 15% by weight of manganese and from 0.05 to 5% by weight of alkali metal, in particular sodium, in each case calculated as oxide.

The reaction temperatures in the hydrogenation are generally from 40 to 150° C., preferably from 40 to 120° C.

The absolute pressure in the hydrogenation is generally from 40 to 250 bar, preferably from 100 to 210 bar.

### Isolation of the XDA:

After the hydrogenation, the ammonia used is, if appropriate, distilled off. Part of the XDA (preferably the amount corresponding to the amount of PN which was fed in) is, if appropriate, discharged and passed to purification. The remaining amount is reused as solvent.

Purification of the xylylenediamine is preferably carried out by distilling off lower-boiling by-products overhead and separating off higher-boiling impurities at the bottom in a distillation.

Particular preference His given to a mode of operation in which, after the hydrogenation, any ammonia and any low-boiling by-products are distilled off overhead and higher-boiling impurities are then separated off from the xylylenediamine at the bottom in a distillation with pure xylylenediamine being obtained via a liquid or gaseous side offtake stream.

Depending on the desired purity, the product (XDA) is additionally extracted with an organic solvent, preferably an aliphatic hydrocarbon, in particular a cycloaliphatic hydrocarbon, very particularly preferably cyclohexane or methylcyclohexane.

This purification by extraction can, for example, be carried out as described in DE-A-1 074 592 (BASF AG).

The hydrogenation to form MXDA can, for example, be carried out in a plant as shown in FIG. 1. MXDA or crude MXDA (stream [2]) is placed in a stirred vessel and heated. IPN (stream [1]) is fed in while stirring. A 15% strength solution of IPN in MXDA is obtained. This solution (stream [3]) is then mixed continuously with ammonia (stream [4]) and preheated together with fresh hydrogen (stream [5]) and, if appropriate, recirculated hydrogen (stream [9]) in the heat exchanger W 300 and fed to the hydrogenation reactor C 300. There, the catalytic hydrogenation to form MXDA occurs, with space velocity and temperature being set so that complete conversion is achieved. The reaction product mixture is cooled and separated from the gas in the high-pressure separator B 301. The gas is circulated by means of compressor V 300 (stream [9]) and part is discharged (stream [10]) to avoid the accumulation of inerts. The liquid phase from B 301 can be partly circulated (stream [6]) or all of it can be passed to the pressure distillation in K 300 in which ammonia is recovered in liquid form (stream [12]) and can be used again in place of fresh-ammonia as stream [4]. Crude MXDA (stream [13]) is obtained at the bottom of the pressure, column K 300 and this comprises, depending on the distillation conditions, only traces of ammonia. It can be used directly and without a further work-up step in place of the pure MXDA (stream [2]) for dissolving a fresh batch of IPN. Part of the crude MXDA can be passed to the purifying distillation in order to obtain MXDA having a purity of >99% by weight. This pure MXDA can likewise be used for dissolving IPN, but preference is given to using crude MXDA to keep the out-lay for distillation small.

### EXAMPLES

### Example 1

A reactor having a reactor volume of 70 ml which is suitable for upflow mode operation was charged with an all-active cobalt catalyst, doped with Mn, P, Na) as 4, mm extrudates. A 15% strength solution (at 60° C.) of IPN in MXDA was fed in at the lower end of the reactor. Hydrogen, and ammonia were likewise fed in from the bottom. At an inflow of 126 g/h of nitrile/MXDA solution and 54 g/h of ammonia, a hydrogen flow of 20 l/h (volume under standard conditions) and a recycle stream of 3.5 ml/min. were set. The reactor pressure was 190 bar (abs.). After ˜150 g of IPN had been reacted at a selectivity of 88% (based on IPN used), 15% of the crude MXDA obtained was discharged. The remaining amount was used as solvent for a further ˜150 g of IPN. This procedure was repeated 10 times. In all cases, no IPN could be detected in the output. The purity of the crude MXDA obtained was 89% by weight after the 10th pass. This corresponds to a selectivity of ˜87% based on IPN used.

### Example 2

Solutions of 15% by weight of IPN in MXDA were prepared batchwise in a stirred vessel at 60° C. and pumped to an intermediate vessel. At the beginning of the campaign, MXDA having a purity of >99% by weight was available. The solution was compressed to 200 bar by means of a high-pressure pump and admixed with liquid ammonia (50 mol of NH₃ per mol of IPN). The mixture was heated to 70° C. and fed-together with hydrogen to a hydrogenation reactor. The reactor was operated adiabatically in a single pass in the downflow mode at a space velocity over the catalyst of 0.3 kg of IPN/I/h. As a result of the heart of reaction, the temperature in the reactor increased to about 100° C. at the outlet. The reaction product mixture was depressurized to about 14 bar and ammonia was distilled off at this pressure and was reused after condensation. The remaining bottom product (=crude MXDA) was used in its entirety without a further work-up-step for dissolving a further batch of IPN which was then hydrogenated. In this way, the crude MXDA was recirculated five times for dissolving IPN before it was finally passed to the purifying distillation. The selectivity based on IPN used was 93%.

1. A process for preparing o-, m- or p-xylylenediamine by hydrogenation of o-, m- or p-phthalonitrile in the presence of a heterogeneous catalyst, which comprises:
feeding a solution of the phthalonitrile in the corresponding isomer of crude xylylenediamine into a hydrogenation reactor, with the crude xylylenediamine having a purity in the range from 85 to 99.7% by weight and a content of higher boilers in the range from 0.3 to 15% by weight and part of the output from the reactor being recirculated to the reactor inlet for hydrogenation in recycle mode.

2. The process according to claim 1 for preparing meta-xylylenediamine by hydrogenation of isophthalonitrile.

3. The process according to claim 1, wherein a 7.5 to 25% strength by weight solution of the phthalonitrile is used.

4. The process according to claim 1, wherein the solution of the phthalonitrile is prepared at a temperature in the range from 40 to 120° C.

5. The process according to claim 1, wherein the solution of the phthalonitrile is prepared at an absolute pressure in the range from 1 to 20 bar.

6. The process according to claim 1, wherein the hydrogenation is carried out in the absence of a further solvent.

7. The process according to claim 1, wherein the hydrogenation is carried out in the presence of ammonia.

8. The process according to claim 1, wherein the hydrogenation is carried out at a temperature in the range from 40 to 150° C.

9. The process according to claim 1, wherein the crude xylylenediamine used as solvent has a purity in the range from 89 to 99.5% by weight and a content of higher boilers in the range from 0.5 to 11% by weight.

10. The process according to claim 1, wherein the crude xylylenediamine used as solvent has been obtained by hydrogenation of phthalonitrile.

11. The process according to claim 1, wherein the crude xylylenediamine used as solvent has a content of lower boilers in the range from 0.01 to 2% by weight and an ammonia content in the range from 0 to 5% by weight.

12. The process according to claim 1 which is carried out continuously.

13. The process according to claim 1, wherein part of the output from the reactor is recirculated continuously as a liquid recycle stream to the reactor inlet (recycle mode).

14. The process according to claim 1, wherein the hydrogenation is carried out over a catalyst comprising Ni, Co and/or Fe as all-active catalyst or on an inert support.

15. The process according to claim 1, wherein the hydrogenation is carried out over an all-active manganese-doped cobalt catalyst.

16. The process according to claim 1, wherein, after the hydrogenation, any ammonia and any lower-boiling by-products are distilled off overhead and part of the crude xylylenediamine obtained is used for preparing the solution of the phthalonitrile used in the process.

17. The process according to claim 1, wherein, after the hydrogenation, a purification of the xylylenediamine is carried out by distilling off any ammonia and any low-boiling by-products overhead and carrying out a removal of higher-boiling impurities at the bottom in a distillation.

18. The process according to claim 1, wherein the xylylenediamine is extracted with an organic solvent to purify it further after the distillation.

19. The process according to claim 1, wherein cyclohexane or methylcyclohexane is used for the extraction.

20. The process according to claim 2, wherein a 7.5 to 25% strength by weight solution of the phthalonitrile is used.
